(19) United States
(12) Patent Application Publication
(10) Pub. No.: US 2005/0107823 A1
(43) Pub. Date: May 19, 2005

(54) Anchored stent and occlusive device for treatment of aneurysms

(76) Inventors: Jim E. Leone, Miami, FL (US); Eric W. Leopold, Redwood City, CA (US)

Correspondence Address: FULWIDER PATTON LEE & UTECHT, LLP;HOWARD HUGHES CENTER, 6060 CENTER DRIVE, TENTH FLOOR, LOS ANGELES, CA, 90045, US

(21) Appl. No.: 10/718,500

(22) Filed: Nov. 19, 2003

(51) Int. Cl. A61M 29/00

(52) U.S. Cl. 606/200

(57) ABSTRACT

This invention relates generally to vasoocclusive devices, and more particularly concerns a vasoocclusive device that has a first elongated, reduced friction configuration in which the vasoocclusive device may be deployed through a catheter or cannula to an anatomical cavity at a site in the vasculature to be treated. The device has a second configuration assumed at the site to be treated which consists of a component that creates an anchor in the adjacent vasculature and a component for framing or filling the anatomical cavity.

### BACKGROUND OF THE INVENTION

1. Field of the Invention

This invention relates generally to vasoocclusive devices, and more particularly concerns a vasoocclusive device that has a first elongated, reduced friction configuration in which the vasoocclusive device may be deployed through a catheter or cannula to an anatomical cavity at a site in the vasculature to be treated, and that has a three dimensional second configuration assumed by the vasoocclusive device at the site to be treated for filling and reinforcing the anatomical cavity.

2. Description of Related Art

The art and science of interventional therapy and surgery has continually progressed towards treatment of internal defects and diseases by use of ever smaller incisions or access through the vasculature or body openings in order to reduce the trauma to tissue surrounding the treatment site. One important aspect of such treatments involves the use of catheters to place therapeutic devices at a treatment site by access through the vasculature. Examples of such procedures include transluminal angioplasty, placement of stents to reinforce the walls of a blood vessel or the like and the use of vasoocclusion devices to treat defects in the vasculature. There is a constant drive by those practicing in the art to develop new and more capable systems for such applications. When coupled with developments in biological treatment capabilities, there is an expanding need for technologies that enhance the performance of interventional therapeutic devices and systems.

One specific field of interventional therapy that has been able to advantageously use recent developments in technology is the treatment of neurovascular defects. More specifically, as smaller and more capable structures and materials have been developed, treatment of vascular defects in the human brain which were previously untreatable or represented unacceptable risks via conventional surgery have become amenable to treatment. One type of non-surgical therapy that has become advantageous for the treatment of defects in the neurovasculature has been the placement by way of a catheter of vasoocclusive devices in a damaged portion of a vein or artery.

Vasoocclusive devices are therapeutic devices that are placed within the vasculature of the human body, typically via a catheter, either to block the flow of blood through a vessel making up that portion of the vasculature through the formation of an embolus or to form such an embolus within an aneurysm stemming from the vessel. The vasoocclusive devices can take a variety of configurations, and are generally formed of one or more elements that are larger in the deployed configuration than when they are within the delivery catheter prior to placement. One widely used vasoocclusive device is a helical wire coil having a deployed configuration which may be dimensioned to engage the walls of the vessels. One anatomically shaped vasoocclusive device that forms itself into a shape of an anatomical cavity such as an aneurysm and is made of a pre-formed strand of flexible material that can be a nickel-titanium alloy is known from U.S. Pat. No. 5,645,558, which is specifically incorporated by reference herein. That vasoocclusive device comprises one or more vasoocclusive members wound to form a generally spherical or ovoid shape in a relaxed state. The vasoocclusive members can be a helically wound coil or a co-woven braid formed of a biocompatible material, and the device is sized and shaped to fit within a vascular cavity or vesicle, such as for treatment of an aneurysm or fistula. The vasoocclusive member can be first helically wound or braided in a generally linear fashion, and is then wound around an appropriately shaped mandrel or form, and heat treated to retain the shape after removal from the heating form. Radiopacity can be provided in the vasoocclusive members by weaving in synthetic or natural fibers filled with powdered radiopaque material, such as powdered tantalum, powdered tungsten, powdered bismuth oxide or powdered barium sulfate.

The delivery of such vasoocclusive devices can be accomplished by a variety of means, including via a catheter in which the device is pushed through the catheter by a pusher to deploy the device. The vasoocclusive devices, which can have a primary shape of a coil of wire that is then formed into a more complex secondary shape, can be produced in such a way that they will pass through the lumen of a catheter in a linear shape and take on a complex shape as originally formed after being deployed into the area of interest, such as an aneurysm. A variety of detachment mechanisms to release the device from a pusher have been developed and are known in the art.

For treatment of areas of the small diameter vasculature such as a small artery or vein in the brain, for example, and for treatment of aneurysms and the like, micro-coils formed of very small diameter wire are used in order to restrict, reinforce, or to occlude such small diameter areas of the vasculature. A variety of materials have been suggested for use in such micro-coils, including nickel-titanium alloys, copper, stainless steel, platinum, tungsten, various plastics or the like, each of which offers certain benefits in various applications. Nickel-titanium alloys are particularly advantageous for the fabrication of such micro coils, in that they can have super-elastic or shape memory properties, and thus can be manufactured to easily fit into a linear portion of a catheter, but attain their originally formed, more complex shape when deployed.

One conventional vasoocclusive coil is known, for example, that has a three dimensional in-filling coil configuration, formed by winding a wire into a helix, and then winding the helix into a secondary form which forms a generally spherical shape, by winding the primary coil about poles placed on winding mandrel. The secondary wound coil is then annealed on the winding mandrel, and the coil is then removed from the winding mandrel and loaded into a carrier for introduction into a delivery catheter. Another similar type of vasoocclusive device is known that can be formed from one or more strands, and can be wound to form a generally spherical or ovoid shape when released and relaxed at the site to be treated. Another implantable vasoocclusive device having multiple secondary layers of primary windings has a final shape that is a generally spherical coil formed of linear or helical primary coils that are wound into a secondary form having three layers. The inner winding is wound and then the second layer formed by winding in the opposite direction of the first layer. The final configuration is a chunky or stepped shape approximately a sphere, ovoid, or egg. Yet another conventional implant for vessel occlusion is made from helical elements of metal or synthetic material by twisting or coiling the elements and forming them into a secondary shape such as a rosette or double rosette for implantation using a catheter, and another vaso-occlusive device is known that has a final conical shape. However, due to the tendency of such three dimensional shaped coils to transform into their expanded, final forms when introduced into a catheter in the body, they are inherently more difficult than a helical coil or a straight wire or micro-cable to push through such a catheter for delivery to a site in the vasculature to be treated, due to friction between the coil and the catheter through which it is delivered to the site to be treated, which can even result in misalignment of the coil within the catheter during delivery.

A growing concern with vasoocclusive device deployment is that once implanted and transformed into their final relaxed forms, they may be subject to migration from the site to be treated. Some vasoocclusive device systems addressing this concern only compound the migration problem by introducing several individual components to form a vasoocclusive device framework that cause more movement within the vasculature during and after deployment. Vasoocclusive devices addressing the migration concerns have consisted of multiple stents interwound in the vasculature, and devices having collapsible framework components connected to stents and embolization elements.

There thus remains a need for a vasoocclusive device that has a three dimensional final form that can be used to frame or fill an anatomical cavity at a site in the vasculature to be treated, reduce or prevent migration of the device after implantation at the site to be treated, and ultimately helps to prevent coil misalignment. The present invention meets these and other needs.

### SUMMARY OF THE INVENTION

Briefly, and in general terms, the present invention provides for an improved vasoocclusive device, that creates a three dimensional shaped coil portion, and a means to anchor the coil portion at the site of the vasculature to be treated. The three dimensional portion will form a vasoocclusive portion for filling the anatomical cavity at the site in the vasculature to be treated. The three dimensional portion of the vasoocclusive coil comprises at least one strand of a flexible material formed to have a first inoperable, substantially linear configuration for insertion into and through a catheter or cannula to a desired portion of the vasculature to be treated, and a second operable configuration for framing or occluding the desired portion of the vasculature to be treated, and further comprising a portion to anchor the occluding portion of the vasoocclusive device in the artery system of the vasculature. This substantially linear configuration allows for reduction of friction of the coil within a catheter or cannula being used to deliver the vasoocclusive device to the site in the vasculature to be treated, and ultimately helps prevent coil realignment or misalignment, or otherwise coil migration after deployment. The vasoocclusive coil may optionally also include a portion having a first inoperable, substantially linear configuration for insertion into and through a catheter or cannula to a desired portion of the vasculature to be treated, and a second operable configuration that is substantially helically shaped, for filling and reinforcing the three dimensional portion, for occluding the desired portion of the vasculature to be treated, in order to combine the best qualities of a three dimensional coil and a helical coil.

The present invention accordingly provides for a vasoocclusive device that is adapted to be inserted into a portion of a vasculature for occluding the portion of the vasculature for use in interventional therapy and vascular surgery. The vasoocclusive device comprises at least one strand of a flexible material formed to have a first inoperable, substantially linear configuration for insertion into and through a catheter or cannula to a desired portion of the vasculature to be treated, and a second operable, three dimensional configuration for framing or occluding the desired portion of the vasculature to be treated. The vasoocclusive device has a portion having a second operable coiled shape for framing or filling the anatomical cavity at the site in the vasculature to be treated, and may optionally include a portion having a second operable, substantially helical shape for framing or filling and reinforcing the desired portion of the vasculature when it is implanted at the site in the vasculature to be treated. The second operable portion advantageously further comprises a portion to anchor the cavity filling portion of the device in the artery system of the vasculature to prevent migration of the device.

In another aspect, the present invention provides for a vasoocclusive device wherein the second operable configuration having an anchor segment further comprises at least one extending loop. The extending loop is curved about a longitudinal axis to form a hollow cylindrical circumferential pattern of loops about the longitudinal axis to provide a contact surface area to anchor the occluding portion of the device adjacent the artery system of the vasculature to be treated.

The present invention also provides a device that is adapted to be inserted into a portion of a vasculature for occluding a portion of the vasculature for use in interventional therapy and vascular surgery. The device comprises at least one strand of a flexible material formed to have a portion with a first inoperable, substantially linear configuration for insertion into and through a catheter to a desired portion of the vasculature to be treated, and a second operable configuration having an anchor segment loaded into the adjacent artery and a coil segment for framing or occluding the desired part of the vasculature to be treated. In a preferred aspect, the second operable configuration having the anchor portion loaded into the adjacent artery and the coil segment further comprises, an inner reinforcement member extending through the coil segment and the anchor portion to reinforce the anchor segment. The inner reinforcement member may be used to aid in secondary shape configurations, and to aid desired stiffness of the coil.

The present invention also provides a method for repairing a portion of a vasculature having a vasoocclusive deformity to restore physiologically normal flow to the portion of the vasculature to be treated. The method comprises the steps of, moving a catheter through the vasculature and to the portion of the vasculature to be treated, moving through the catheter a vasoocclusive device comprising at least one strand of a flexible material formed to have a portion with a first inoperable, substantially linear configuration for insertion into and through a catheter to a desired portion of the vasculature to be treated, and a second operable configuration for framing or occluding the desired portion of the vasculature to be treated, and anchoring a portion of the second operable configuration of the device in the artery system of the vasculature.

These and other features and advantages of the invention will become apparent from the following detailed description and the accompanying drawings, which illustrate by way of example the features of the invention.

### BRIEF DESCRIPTION OF THE DRAWINGS

FIG. 1 is a cross sectional view of a vascular member with an aneurysm illustrating the approach of a vasoocclusive device, in a first inoperable configuration, towards the aneurysm.

FIG. 2 is a side elevational view showing a preferred embodiment of the second operable configuration having an occluding portion and an anchor portion.

FIG. 3 is the vasoocclusive device of FIG. 2, further depicting an anchor portion having fewer extended loops.

FIG. 4 is an illustration of a vasoocclusive device of FIG. 2 deployed within an aneurysm.

FIG. 5 is an illustration of a vasoocclusive device of FIG. 3 deployed within an aneurysm.

FIGS. 6a and 6b are illustrations of a vasoocclusive device formed with a strand of material deployed within an aneurysm at a vasculature bifurcation.

FIG. 7 is the vasoocclusive device of FIG. 2 having an inner reinforcement member attached therein.

FIGS. 8a and 8b are side views showing an alternative embodiment of the second operable configuration having a 3D helical framing portion and an anchor portion.

### DETAILED DESCRIPTION OF THE PREFERRED EMBODIMENTS

As shown in the exemplary drawings, which are provided for the purposes of illustration and not by way of limitation, the device of the present invention is designed to be deployed intravascularly without the necessity of balloons or other expansive elements and can be deployed from a micro-catheter directly into the area to be treated. The intravascular device of the present invention is particularly useful for treatment of damaged arteries incorporating aneurysms and the like, particularly those which are treatable by the use of embolic coils or other embolic devices or agents used to occlude the aneurysm. More particularly, the device of the invention is particularly well adapted to use with the types of catheters used to place such embolic coils in aneurysms, and the device is used to anchor the device adjacent the area of the aneurysm while assisting in the retention of the embolic devices within the dome of the aneurysm.

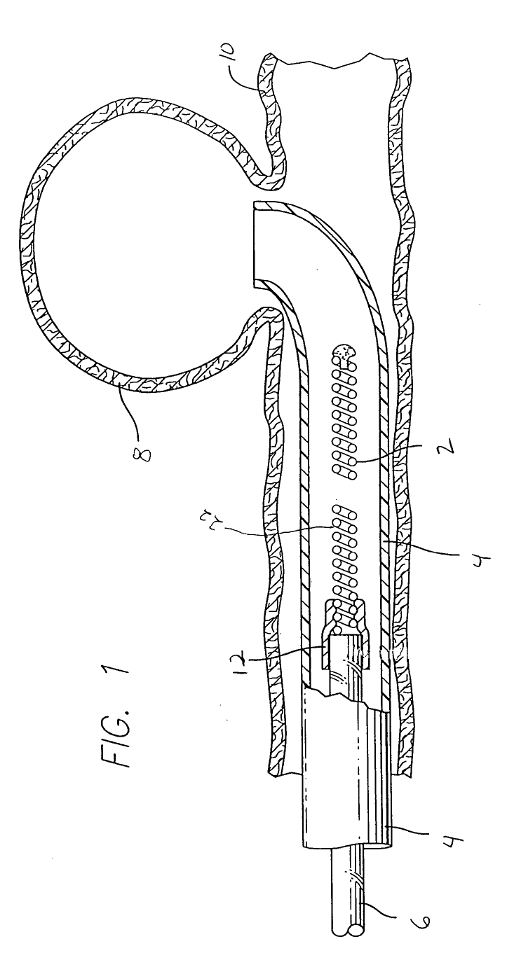

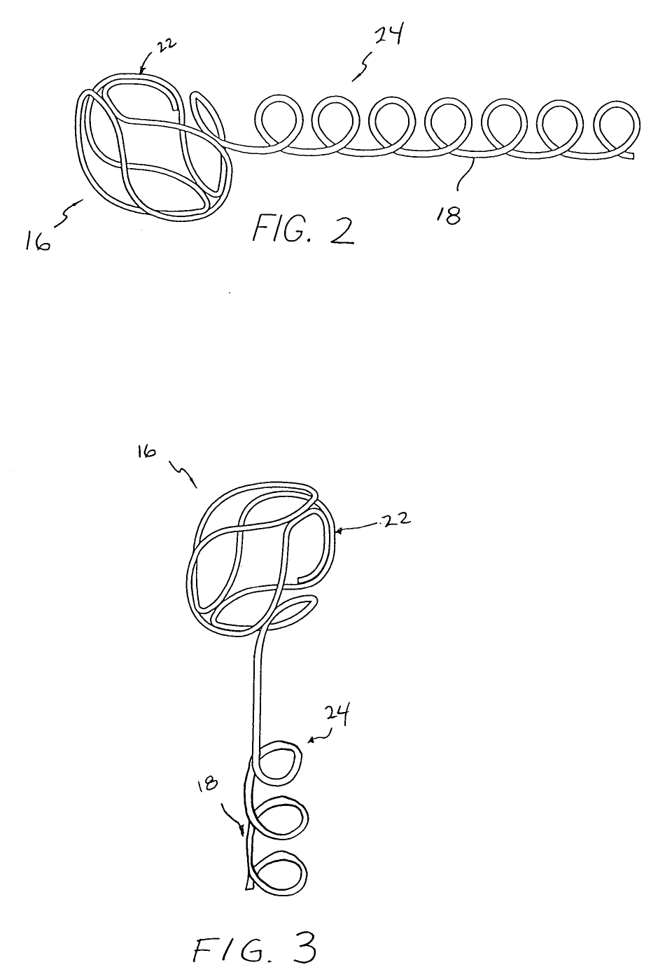

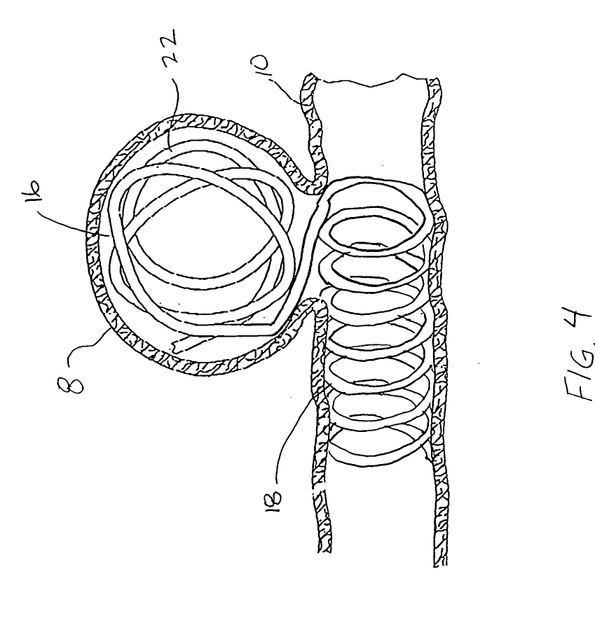

As is illustrated FIGS. 1-8, the invention is accordingly embodied in a vasoocclusive device that is adapted to be inserted into a portion of a vasculature for framing or occluding the portion of the vasculature for use in interventional therapy and vascular surgery. The vasoocclusive device 2 is formed from at least one strand of a flexible material 22 formed to have a first inoperable, substantially linear configuration, as illustrated in FIG. 1, for insertion through a catheter 4 into a desired portion of the vasculature to be treated, such as an aneurysm 8, or other anatomical malformation of the vasculature to be treated, and a second operable configuration illustrated in FIGS. 2-8, for framing or occluding the desired portion of the vasculature to be treated. This substantially linear configuration allows for reduction of friction of the coil within a catheter or cannula being used to deliver the vasoocclusive device to the site in the vasculature to be treated, and ultimately helps prevent coil realignment or misalignment, or otherwise coil migration after deployment.

FIG. 1 illustrates a helically wound vasoocclusive coil 2 which is formed to fit within a catheter for insertion into an area upon which a therapeutic procedure is to be performed. FIG. 1 further shows a catheter pusher member 6, which is detachably attached to a vasoocclusive coil 2 by a collar 12, for delivering the vasoocclusive coil 2 for insertion into an aneurysm 8 projecting laterally from a blood vessel 10. The end of the catheter 4 is typically introduced into the opening of the aneurysm by use of a guide wire (not shown), and the coil and pusher member are introduced into the catheter to insert the vasoocclusive coil into the aneurysm. While those skilled in the art can appreciate that several varieties of vasoocclusive devices and deployment systems exist, the above referenced delivery and deployment system is provided as a general reference and is not intended to be a limitation of the present invention.

As illustrated in FIG. 2, one preferred embodiment of the present invention shows a substantially spherical occlusive device 16 in the operable configuration. The vasoocclusive device 2 comprises at least one strand 22 of flexible material formed to have a first inoperable, substantially linear configuration for insertion into and through a desired portion of a vasculature, and a second operable coiled shape for framing or filling the anatomical cavity, and may optionally include a second operable, substantially spherical shape for occluding at least a portion of the vasculature to be treated. Preferably, the vasculature to be treated is an artery and the desired portion is an aneurysm, however, the invention contemplates that any bodily anatomical cavity may be occluded by the device. The strand 22 shown is wound in a tertiary substantially spherical structure so as to have multiple loops spaced to form a cavity, or cage-like structure. The invention contemplates that the occlusive device 2 is wound into and is self-forming into a substantially spherical or distorted spherical form. By the term “substantially spherical” is meant a shape which includes spherical as well as other distorted shapes, such as ovate, ovoid, or ellipsoid, but in any event having two orthogonal cross sections which are closed shapes having no substantially straight sides.

The invention provides a second operable configuration means for a non-linear portion of the occluding device to engage an artery wall for securing the occluding device in the artery system of the vasculature. As shown in FIG. 2, the means for securing a portion of the vasoocclusive device in the artery system comprises an anchor portion 18 of the second operable configuration 24 to secure the framing or occluding portion 16 of the device and prevent migration of the device after deployment. The anchor portion 18 of the device is dimensioned to engage the walls 10 of the artery system. By the term “framing” is meant to fill the outer portion of an aneurysm or vasculature matter that will be filled inside with additional coil or occlusive materials. It is contemplated that the area to be treated may be framed to be filled with hydrogels, microcellular foam, other therapeutic materials or coils.

The vasoocclusive device of the present invention is formed of at least one strand of a wire 22 that is configured to be a flexible coil. Alternatively, the device may be formed of several flexible wires into other shapes. The vasoocclusive coil 2 may be formed from a wide variety of materials including, but not limited to one or more strands 22 of a metal or metal alloy such as stainless steel or a nickel-titanium alloy, which may include a radiopaque strand. Preferably, the strand 22 is a wire constructed of a radiopaque material such as a metal or a polymer. Suitable metals and alloys for the wiring include Platinum Group metals, especially platinum, rhodium palladium, as well as tungsten, gold, silver, tantalum, and alloys of these metals. Highly preferred is a platinum-tungsten alloy.

The wire may also be of any of a wide variety of stainless steels if some sacrifice of radiopacity may be tolerated. Very desirable materials of construction, from a mechanical point of view, are materials which maintain their shape despite being subjected to high stress. Certain “super-elastic alloys” include nickel-titanium alloys (48-58 atomic % nickel, and optionally containing modest amounts of iron); copper-zinc alloys (38-42 weight % zinc); copper-zinc alloys containing 1-10 weight % of beryllium, silicon, tin, aluminum, or gallium; or nickel-aluminum alloys (36-38 atomic % aluminum). Particularly preferred is the shape memory metal such as nickel titanium alloy, such as that available under the trade name NITINOL. These are very sturdy alloys which will tolerate significant flexing without deformation even when used as a very small diameter wire. Additionally, the strand may be constructed of a polymer, such as polyvinyl alcohol foam, for example.

Generally speaking, when the vasoocclusive device 2 is formed of a metal such as platinum or a super-elastic alloy such as NITINOL, the diameter of the wire used in the production of the coil will be in the range of 0.0005 and 0.006 inches. The wire of such diameter is typically then wound into a coil having a primary diameter of between 0.005 and 0.018 inches. The preferable diameter is 0.009 to 0.018 inches. The wire should be of sufficient diameter to provide a hoop strength to the resulting device sufficient to hold the device 2 in place within the chosen body cavity without distending the wall of the cavity and without moving from the cavity as a result of the repetitive fluid pulsing found in the vascular system. Obviously, should a super-elastic alloy such NITINOL be used, the diameter of the coil wire can be significantly smaller than that used when the relatively ductile platinum or platinum-tungsten alloy is used as the material of construction. Finally, the overall diameter of the device in the operable configuration is generally between 2 and 40 millimeters. Most aneurysms within the cranial vasculature can be treated by one or more devices having those diameters.

Alternatively, the vasoocclusive strand 22 may be adapted with fibers such as synthetic radiolucent fibers or polymers (or metallic threads coated with radiolucent or radiopaque fibers) such as dacron (polyester), polyglycolic acid, polylactic acid, fluoropolymers (polytetrafluoro-ethylene), nylon (polyamide), or even silk. Natural fibers such as silk, cotton or wool may also be employed. Should a fiber be used as the major component of the strand 22, it is desirably filled with some amount of a known radiopaque material such as powdered tantalum powdered tungsten bismuth oxide, barium sulfate, and the like.

In a preferred embodiment of the present invention, the vasoocclusive strand 22 has a secondary structure of helically wound flexible material. The helixes provide further support to the substantially spherical form in the operable condition 24. The helix advantageously provides contact surface area for anchoring the occluding portion 16 of the device in the artery system of the vasculature. The vasoocclusive device 2 may optionally be formed into three dimensional shapes such as, conical, spherical or other geometric shapes.

By way of example, the method of manufacturing the vasoocclusive device of the present invention comprises winding a strand of flexible material onto a mandrel (not shown) suitable for making a substantially spherical vasoocclusive device. The mandrel can primarily consist of a core (not shown). The core is typically made of a refractory material, such as alumina or zirconia. The function of the core is simply to form a support for winding that will not pollute the vasoocclusive device during the heat-treatment step to be described below, and will provide a specific substantially spherical form for the vasoocclusive device during the heat-treatment step. Circumferentially continuous grooves on the surface of the core may be preferably provided to assist in regularly aligning the strand as it is being wound about the core. Additionally, a small strand receptacle may be provided to insert and hold the end or ends of the strand in place when performing the heating step. Other methods of winding a strand around a core will be apparent to those skilled in the art. The continuous grooves are preferably provided to permit the strand to be wound about the core with minimal kinking or angulation of the coils.

If the entire then-wound vasoocclusive device is metallic, it may be placed in an oven at an appropriate temperature to “set” or impart the substantially spherical form to the device. If the device is a platinum alloy or of nitinol, such a temperature is 1100 degrees Fahrenheit, for 4 hours to provide a modest amount of preshaping to the resulting vasoocclusive device. Should the make-up of the vasoocclusive device not be solely metal, in that it contains readily meltable plastic or the like, the temperature at which the heat treatment takes place is significantly lower and typically for a significantly shorter period of time. The flexural modulus of most plastics being significantly lower than that of metals, the bulk of the polymer-based device will be significantly larger than that of the metal-based device.

After cooling, the device is removed from the core. The vasoocclusive device is then placed in a cannula or catheter for delivery in the inoperable substantially linear configuration into a selected body cavity or vesicle, where it then assumes the operable substantially spherical configuration.

Practitioners in this medical device area will undoubtedly have other ways of producing the noted anatomically shaped occlusive and vasoocclusive devices. Briefly, the inventive devices are typically supplied in a prepackaged form in a sterile cannula which is adapted to engage the proximal end of a catheter. Once the catheter is in place within a vessel and the distal end of the catheter is placed into, e.g., a mouth of an aneurysm, the vasoocclusive device is inserted into the aneurysm, where it assumes its relaxed shape. Although the device may be used with a flexible pusher without connection to the vasoocclusive device described here, much more desirable is the use of a detachable coupling on the vasoocclusive device and the pusher. Any of the detachable couplings described above in the Background of the Invention or other detachable couplings would be suitable in this instance.

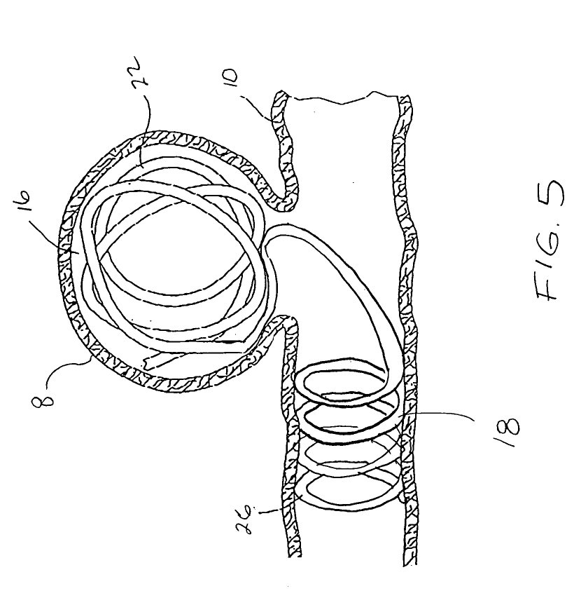

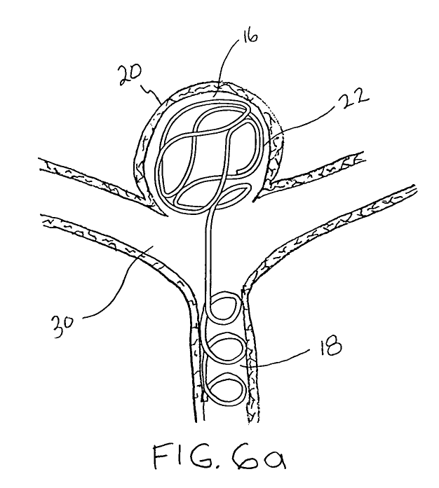

In another preferred embodiment, as shown in FIGS. 4 and 5, it is intended that the vasoocclusive device 2 in the operable configuration 24 be in a roughly spherical cavity or cage-like structure where at least 90-95% of the strand 22 is in the outer 10-15% of the diameter of the device 2. The precise number of loops of the strand will vary and depends upon the type of anatomical cavity to be filled, and upon the length of catheter tubing necessary for deployment in the extended, linear position. In a preferred aspect, the vasoocclusive device is not totally inserted into the aneurysm, where it assumes its relaxed shape, a portion of the strand, forming the anchor portion 18, remains outside of the spherical cavity to create the contact surface area for anchoring the device in the artery system of the vasculature.

Referencing FIG. 5, in another presently preferred embodiment, the invention provides for a vasoocclusive device 2 wherein the second operable configuration 24 having an anchor segment 18 loaded into the adjacent artery, further comprises at least one extending loop 26 extending along a longitudinal axis into the vasculature from a position proximal to a position distal of the aneurysm to be treated. A single piece of shape memory or superelastic alloys such as nickel-titanium alloy, may be wound over an essentially cylindrical mandrel into which are formed channels representing a progressive loop configuration of the invention. The extending loop 26 is curved about a longitudinal axis to form a hollow cylindrical circumferential pattern of loops about the longitudinal axis to provide a contact surface area to anchor the occluding portion of the device adjacent the artery system of the vasculature to be treated.

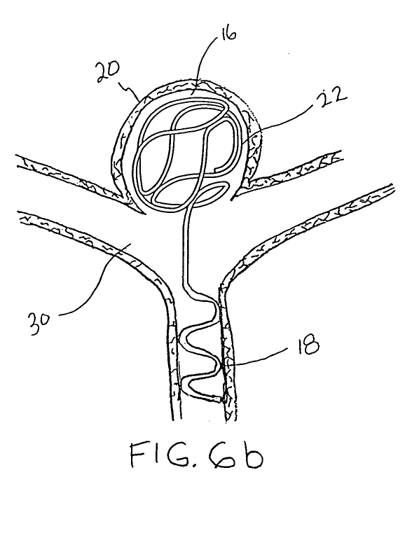

In another preferred embodiment, as illustrated in FIG. 6a, the at least one strand of a flexible material formed to have a portion with a first inoperable, substantially linear configuration, and a second operable configuration having a coil segment for occluding the desired part of the vasculature to be treated, and an anchor segment 18 loaded into the adjacent artery, may be configured to be inserted into a bifurcation aneurysm 20. The anchor segment 18 may be configured to provide a linear wire extending from the occluding coil segment 16 at the bifurcated portion 30 to the anchor segment 18 loaded into the adjacent artery. At least one extending loop is formed at the anchor segment to engage the wall of the artery and secure the device. An advantage of this vasoocclusive device is that blood flow through the bifurcated area will be minimally affected by the device. Some vasoocclusive devices implanting multiple coils in the bifurcation branches may impede blood flow therein. As shown in FIG. 6b, another aspect of the presently preferred invention includes an anchor segment 18 configured to provide a linear wire extending from the occluding coil segment 16 to a sinusoidal shaped anchor segment 18 loaded into the adjacent artery and engaging the wall therein.

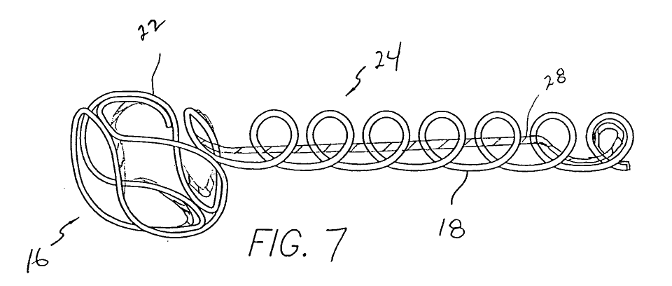

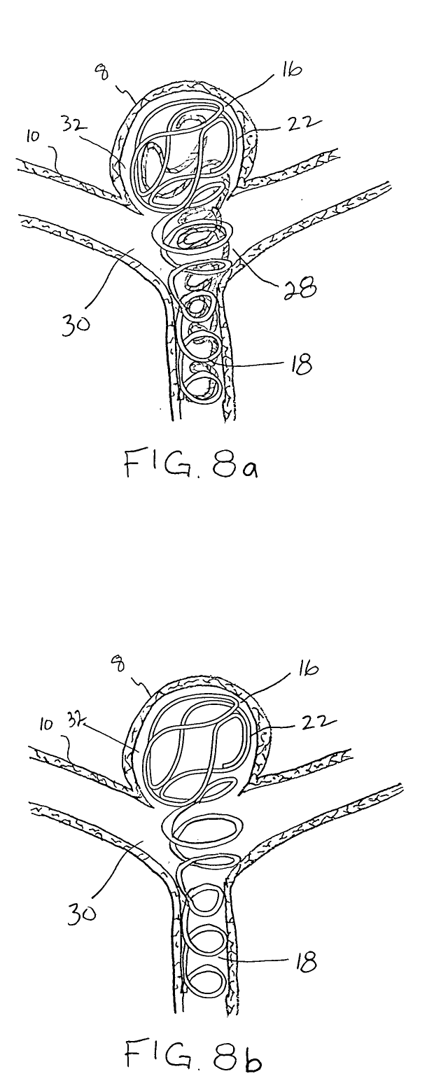

As illustrated in FIGS. 7 and 8, in still another presently preferred embodiment, the second operable configuration 24 further comprises, an inner reinforcement member 28 extending through the primary wind of the coil segment 16 and the anchor portion 18 to reinforce the anchor within the vasculature. Attachment of the inner reinforcement member 28 further secures the embolic coil or other embolic occlusive or other vasoocclusive devices deployed in an aneurysm. The vasoocclusive coil is reinforced by the inner reinforcement member that is fixedly attached therein. Optionally, the reinforcement member 28 may be fixedly attached at one end at or near a distal end of the vasoocclusive coil, and may be detachably mounted at the other end of the vasoocclusive coil to an elongated pusher member to allow for placement and release of the vasoocclusive coil within the patient's vasculature. The inner reinforcement member 28 may be detachably mounted to the distal end of the pusher member (not shown), for example, by at least one loop of fiber material, by a displaced coil at the proximal end of the vasoocclusive coil, or by a loop attached at the proximal end of the vasoocclusive device as a socket. The inner reinforcement member 28 may be formed as a ribbon, wire, braid, primary wind, or stranded material and may be formed from a therapeutic non-metallic material. The inner reinforcement member may also be formed from a metal or metal alloy, which may be a radiopaque metal, such as platinum, tungsten and gold, or a shape memory material, such as NITINOL.

The inner reinforcement member 28 may be used to aid in forming the vasoocclusive device secondary shape configurations, and to aid desired stiffness of the coil.

The invention also provides for an inner reinforcement member configured of multiple stands forming double strand of a plurality of sinusoidal loops. The sinusoidal loop configuration having upper and lowers arcs that extend along a longitudinal axis. The sinusoidal loops are curved about the longitudinal axis to form a generally hollow cylindrical circumferential pattern of loops about the longitudinal axis. In one aspect, the sinusoidal loops may have varying dimensions, varying loop spacing, or more tightly coiled loops to thereby provide a greater contact surface area for anchoring of vasoocclusive devices within the vasculature. In another preferred aspect, FIG. 8a illustrates the second operable configuration having a helical framing portion 32 and an anchor portion 18, further including an inner reinforcement member helically wound opposite the vasoocclusive device, thereby forming transverse loops providing enhanced stiffness and anchoring reinforcement. In still another preferred aspect, the inner reinforcement member coil winding pitch may be adjusted to increase or decrease the stiffness of the transverse loops. FIG. 8b illustrates the vasoocclusive device of FIG. 8a, having a framing portion 32 and an anchor portion 18 without the inner reinforcement member.

It will be apparent from the foregoing that while particular forms of the invention have been illustrated and described, various modifications can be made without departing from the spirit and scope of the invention. Accordingly, it is not intended that the invention be limited, except as by the appended claims.

1. A vasoocclusive device that is adapted to be inserted into a portion of a vasculature for occluding a portion of the vasculature for use in interventional therapy and vascular surgery, comprising:
at least one strand of a flexible material formed to have a first inoperable, substantially linear configuration for insertion into and through a catheter to a desired portion of the vasculature to be treated, and a second operable configuration for framing or occluding the desired part of the vasculature to be treated, said operable configuration including a first portion configured to frame or occlude a part of the vasculature to be treated and a second non linear portion configured to engage an artery wall for securing the occluding device in the artery system of the vasculature.

2. The vasoocclusive device of claim 1, wherein the portion for securing the occluding device in an artery system of the vasculature comprises an anchor portion of the second operable configuration to secure the occluding portion of the device in the artery system of the vasculature.

3. The vasoocclusive device of claim 2, wherein the anchor portion comprises a plurality of extending loops along a longitudinal axis to thereby provide contact surface area for anchoring the occluding portion of the device in the artery system of the vasculature.

4. The vasoocclusive device of claim 1, further comprising a second portion having a first inoperable, substantially linear configuration for insertion into and through a catheter to a desired portion of the vasculature to be treated, and a second operable, coiled shape for filling and reinforcing the desired portion of the vasculature when the vasoocclusive device is implanted at the site in the vasculature to be treated.

5. The vasoocclusive device of claim 1, further comprising a second portion having a first inoperable, substantially linear configuration for insertion into and through a catheter to a desired portion of the vasculature to be treated, and a second operable, substantially helical coil shape for filling and reinforcing the desired portion of the vasculature when the vasoocclusive device is implanted at the site in the vasculature to be treated.

6. The vasoocclusive device of claim 1, wherein said at least one strand of a flexible material is a helical shape.

7. The vasoocclusive device of claim 1, wherein said at least one strand of a flexible material is a wire.

8. The vasoocclusive device of claim 1, wherein said flexible material comprises an alloy of titanium and nickel.

9. The vasoocclusive device of claim 1, wherein said flexible material comprises a metal selected from the group consisting of platinum, palladium, rhodium, gold, tungsten, and alloys thereof.

10. The vasoocclusive device of claim 1, wherein said vasoocclusive device is formed from at least one flexible strand of a resilient radiopaque material to provide a radiopaque marker of the deployed configuration of a device made of the strand during vascular surgery.

11. The vasoocclusive device of claim 10, wherein said radiopaque strand comprises an alloy selected from the group consisting of platinum, tungsten and gold.

12. The vasoocclusive device of claim 1, wherein said at least one strand comprises a super-elastic material.

13. The vasoocclusive device of claim 12, wherein said super-elastic material comprises a nickel-titanium alloy.

14. The vasoocclusive device of claim 1, wherein said at least one strand comprises a shape memory material.

15. The vasoocclusive device of claim 14, wherein said shape memory material comprises a nickel-titanium alloy.

16. The vasoocclusive device of claim 1, wherein the anchor portion is formed to reinforce the vessel in the vicinity of the damaged portion of the vasculature to be treated.

17. A vasoocclusive device that is adapted to be inserted into a portion of a vasculature for occluding a portion of the vasculature for use in interventional therapy and vascular surgery, comprising:
at least one strand of a flexible material formed to have a first inoperable, substantially linear configuration for insertion into and through a catheter to a desired portion of the vasculature to be treated, and a second operable configuration having an anchor segment loaded into the adjacent artery and a coil segment for framing or occluding the desired part of the vasculature to be treated, said operable configuration including a first portion configured to frame or occlude a part of the vasculature to be treated and a second non-linear portion configured to engage an artery wall for securing the occluding device in the artery system of the vasculature; and
wherein said anchor portion is formed to reinforce the vessel in the vicinity of the damaged portion of the vasculature to be treated.

18. The vasoocclusive device of claim 17, the second operable configuration having an anchor segment further comprises at least one extending loop, the extending loop being curved about a longitudinal axis to form a hollow cylindrical circumferential pattern of loops about the longitudinal axis to provide a contact surface area to anchor the occluding portion of the device adjacent the artery system of the vasculature to be treated.

19. The vasoocclusive device of claim 17, wherein the second portion having a first inoperable, substantially linear configuration for insertion into and through a catheter to a desired portion of the vasculature to be treated, and a second operable configuration consisting of a coil segment further comprising, a coiled shape for filling and reinforcing the desired part of the vasculature when the vasoocclusive device is implanted at the site in the vasculature to be treated.

20. The vasoocclusive device of claim 17, further comprising a second portion having a first inoperable, substantially linear configuration for insertion into and through a catheter to a desired portion of the vasculature to be treated, and a second operable, substantially helical coil shape for filling and reinforcing the desired portion of the vasculature when the vasoocclusive device is implanted at the site in the vasculature to be treated.

21. The vasoocclusive device of claim 17, wherein said at least one strand of a flexible material is a helical shape.

22. The vasoocclusive device of claim 17, wherein said at least one strand of a flexible material is a wire.

23. The vasoocclusive device of claim 17, wherein said flexible material comprises an alloy of titanium and nickel.

24. The vasoocclusive device of claim 17, wherein said flexible material comprises a metal selected from the group consisting of platinum, palladium, rhodium, gold, tungsten, and alloys thereof.

25. The vasoocclusive device of claim 17, wherein said vasoocclusive device is formed from at least one flexible strand of a resilient radiopaque material to provide a radiopaque marker of the deployed configuration of a device made of the strand during vascular surgery.

26. The vasoocclusive device of claim 25, wherein said radiopaque strand comprises an alloy selected from the group consisting of platinum, tungsten and gold.

27. The vasoocclusive device of claim 17, wherein said at least one strand comprises a super-elastic material.

28. The vasoocclusive device of claim 27, wherein said super-elastic material comprises a nickel titanium alloy.

29. The vasoocclusive device of claim 17, wherein said at least one strand comprises a shape memory material.

30. The vasoocclusive device of claim 29, wherein said shape memory material comprises a nickel-titanium alloy.

31. The vasoocclusive device of claim 17, wherein the second portion having a first inoperable, substantially linear configuration for insertion into and through a catheter to a desired portion of the vasculature to be treated, and a second operable configuration having an anchor portion loaded into the adjacent artery and a coil segment, further comprising an inner reinforcement member extending through the coil segment and the anchor portion to reinforce the anchor segment.

32. The vasoocclusive device of claim 17, wherein the second portion having a first inoperable, substantially linear configuration for insertion into and through a catheter to a desired portion of the vasculature to be treated, and a second operable configuration having an anchor portion loaded into the adjacent artery and a coil segment, further comprising an inner reinforcement member extending through the anchor portion to reinforce the anchor segment.

33. The vasoocclusive device of claim 31, wherein said inner reinforcement member is fixedly attached at the anchor portion of the vasoocclusive device.

34. The vasoocclusive device of claim 31, wherein said reinforcement member is a coil shape.

35. The vasoocclusive device of claim 31, wherein the reinforcement member is helically wound opposite the formed flexible material.

36. The vasoocclusive device of claim 31, wherein the reinforcement member is formed of a ribbon.

37. The vasoocclusive device of claim 31, wherein the reinforcement portion is formed of a wire.

38. The vasoocclusive device of claim 31, wherein the reinforcement portion is formed of a tapered wire.

39. The vasoocclusive device of claim 31, wherein said inner reinforcement member further comprises transverse loops.

40. A vasoocclusive device that is adapted to be inserted into a portion of a vasculature for occluding a portion of the vasculature for use in interventional therapy and vascular surgery, comprising:
at least one strand of a flexible material formed to have a first inoperable, substantially linear configuration for insertion into and through a catheter to a desired portion of the vasculature to be treated, and a second operable configuration for framing or occluding the desired site of the vasculature to be treated, said operable configuration including a first portion configured to frame or occlude a part of the vasculature to be treated and a second non-linear portion configured to engage an artery wall for securing the occluding device in the artery system of the vasculature, wherein the operable configuration for framing or occluding the desired site of the vasculature further comprises at least one extending loop to anchor the occluding portion of the device in the artery system of the vasculature.

41. The vasoocclusive device of claim 40, further comprising a second portion having a first inoperable, substantially linear configuration for insertion into and through a catheter to a desired portion of the vasculature to be treated, and a second operable, coiled shape for filling and reinforcing the desired portion of the vasculature when the vasoocclusive device is implanted at the site in the vasculature to be treated.

42. The vasoocclusive device of claim 40, further comprising a second portion having a first inoperable, substantially linear configuration for insertion into and through a catheter to a desired portion of the vasculature to be treated, and a and through a catheter to a desired portion of the vasculature to be treated, and a second operable, substantially spherical configuration for occluding at least a portion of said vasculature to be treated, said substantially spherical configuration having about 90% of said strand in about the outer 15% of the diameter of said substantially spherical configuration.

43. The vasoocclusive device of claim 40, further comprising a second portion having a first inoperable, substantially linear configuration for insertion into and through a catheter to a desired portion of the vasculature to be treated, and a second operable, substantially helical coil shape for filling and reinforcing the desired portion of the vasculature when the vasoocclusive device is implanted at the site in the vasculature to be treated.

44. The vasoocclusive device of claim 40, wherein said at least one strand of a flexible material is a helical shape.

45. The vasoocclusive device of claim 40, wherein said at least one strand of a flexible material is a wire.

46. The vasoocclusive device of claim 40, wherein said flexible material comprises an alloy of titanium and nickel.

47. The vasoocclusive device of claim 40, wherein said flexible material comprises a metal selected from the group consisting of platinum, palladium, rhodium, gold, tungsten, and alloys thereof.

48. The vasoocclusive device of claim 40, wherein said vasoocclusive device is formed from at least one flexible strand of a resilient radiopaque material to provide a radiopaque marker of the deployed configuration of a device made of the strand during vascular surgery.

49. The vasoocclusive device of claim 40, wherein said at least one strand comprises a super-elastic material.

50. The vasoocclusive device of claim 49, wherein said super-elastic material comprises a nickel titanium alloy.

51. The vasoocclusive device of claim 40, wherein said at least one strand comprises a shape memory material.

52. The vasoocclusive device of claim 51, wherein said shape memory material comprises a nickel-titanium alloy.

53. The vasoocclusive device of claim 40, wherein the anchor portion is formed to reinforce the vessel in the vicinity of the damaged portion of the vasculature to be treated.

54. A method for repairing a portion of a vasculature having a vasoocclusive deformity to restore physiologically normal flow to the portion of the vasculature to be treated, comprising the steps of:
moving a catheter through the vasculature and to the portion of the vasculature to be treated;
moving through said catheter a vasoocclusive device comprising at least one strand of a flexible material formed to have a first inoperable, substantially linear configuration for insertion into and through a catheter to a desired portion of the vasculature to be treated, and a second operable configuration for framing or occluding the desired portion of the vasculature to be treated, and anchoring a portion of said second operable configuration of the device in the artery system of the vasculature.

55. The method of claim 54, wherein said anchor portion comprises a plurality of extending loops along a longitudinal axis to thereby provide contact surface area for anchoring the occluding portion of the device in the artery system of the vasculature.
